(19) United States
(12) Patent Application Publication
(10) Pub. No.: US 2012/0220776 A1
(43) Pub. Date: Aug. 30, 2012

(54) PROCESS FOR THE PREPARATION OF ILOPERIDONE USING A NOVEL INTERMEDIATE

(75) Inventors: Dodda Mohan Rao, Hyderabad (IN); Pingili Krishna Reddy, Hyderabad (IN); Kanakuntla Chandana Reddy, Hyderabad (IN); Mohammad Rizwana, Hyderabad (IN)

(73) Assignee: SYMED LABS LIMITED, Hyderabad, Andhra Pradesh (IN)

(21) Appl. No.: 13/505,577

(22) PCT Filed: Nov. 19, 2009
(86) PCT No.: PCT/IN09/00663

§ 371 Date: May 2, 2012

(30) Foreign Application Priority Data
Nov. 19, 2009 (IN) PCT/IN2009/000663

(51) Int. Cl. C07D 261/20; C07C 43/205; C07C 45/29

(52) U.S. Cl. 546/198; 568/315; 568/649

(57) ABSTRACT

The present invention provides a novel process for the preparation of iloperidone using a novel intermediate. Thus, for example, 4-(3-chloropropoxy)-3-methoxybenzaldehyde is reacted with methyl magnesium iodide in ether and the reaction mass is heated for 6 hours at reflux temperature, the resulting mass is cooled to ambient temperature and then poured into a mixture of ice, water and dilute hydrochloric acid to produce 1-[4-(3-chloropropoxy)-3-methoxyphenyl]ethanol, which is then subsequently converted to iloperidone.

### FIELD OF THE INVENTION

The present invention relates to a novel process for the preparation of iloperidone using a novel intermediate.

### BACKGROUND OF THE INVENTION

EP Patent No. 402644 discloses N-(aryloxyalkyl)heteroarylpiperidines and heteroarylpiperazines. The compounds are antipsychotic agents. Among them, iloperidone, chemically 1-[4-[3-[4-(6-Fluoro-1,2-benzisoxazol-3-yl)-1-piperidinyl]propoxy]-3-methoxyphenyl]ethanone is a most important antipsychotic agent. Iloperidone is represented by the following structure of formula I:

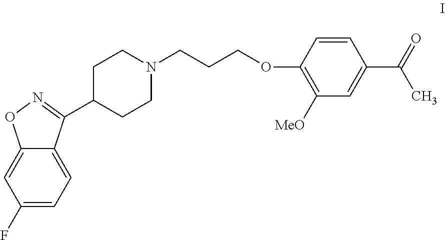

Various processes for the preparation of iloperidone or a pharmaceutically acceptable salt thereof and its intermediates are disclosed in EP 402644, EP 542136 and J. Med. Chem. 38, 1995, 1119-1131.

In the preparation of iloperidone, 1-[4-(3-halopropoxy)-3-methoxyphenyl]ethanone of formula II:

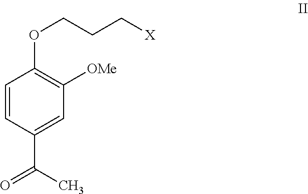

is a key intermediate.

According to the processes described in the prior art iloperidone is prepared by reacting 1-[4-(3-chloropropoxy)-3-methoxyphenyl]-ethanone with 6-fluoro-3-(4-piperidinyl)-1,2-benzisoxazole hydrochloride in the presence of an inorganic base.

The processes for the preparation of 1-[4-(3-chloropropoxy)-3-methoxyphenyl]-ethanone described in the prior art are associated with many drawbacks.

As per the processes exemplified in the EP 542136 and J. Med. Chem. 38, 1995, 1119-1131, the 1-[4-(3-chloropropoxy)-3-methoxyphenyl]-ethanone is prepared by reacting 4-(3-chloropropoxy)-3-methoxybenzaldehyde with a reaction mass containing a stirred suspension under nitrogen of sodium hydride in tetrahydrofuran and a solution of pyrazole in tetrahydrofuran to produce 4-(3-chloropropoxy)-3-methoxybenzoic acid. The resulting benzoic acid compound is then reacted with thionyl chloride in dichloromethane to produce 4-(3-chloropropoxy)-3-methoxybenzoyl chloride, which is then reacted with methyl bromide in methylene chloride in the presence of hexamethylphosphoroustriamide at a temperature of −70° C. to −65° C. to produce the 1-[4-(3-chloropropoxy)-3-methoxyphenyl]-ethanone.

The processes for the preparation of 1-[4-(3-chloropropoxy)-3-methoxyphenyl]-ethanone described in the aforementioned prior art suffer from the disadvantages such as the use of explosive and difficult to handle reagents like sodium hydride, highly hazardous materials like thionyl chloride and methyl bromide, additional reagents like pyrazole and hexamethylphosphoroustriamide, and highly inflammable solvents like hexane. Moreover, the prior art processes are very lengthy, and involve the use of tedious and cumbersome procedures like low temperatures (−70° C. to −65° C.), column chromatographic purifications, multiple isolations/recrystallizations, and thus resulting in a poor product yield.

Based on the aforementioned drawbacks, the prior art processes have been found to be unsuitable for the preparation of 1-[4-(3-halopropoxy)-3-methoxyphenyl]ethanone of formula II at lab scale and in commercial scale operations.

We have discovered a novel process for the preparation of 1-[4-(3-halopropoxy)-3-methoxyphenyl]ethanone of formula II with high yields and purity using a novel intermediate. The novel process avoids the tedious and cumbersome procedures of the prior process, and thereby resolving the problems associated with the processes described in the prior art, and which is more convenient to operate at lab scale and in commercial scale operations.

### SUMMARY OF THE INVENTION

In accordance with one aspect of the present invention, there is provided a novel process for the preparation of iloperidone intermediate of formula II:

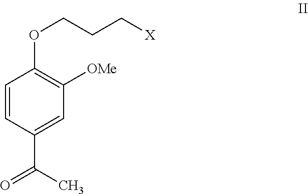

wherein X is selected from F, Br, Cl and I;
which comprises:

a) reacting an aldehyde compound of formula III:

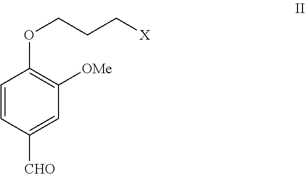

wherein X is as defined above;
with methyl magnesium halide to produce a hydroxy compound of formula IV:

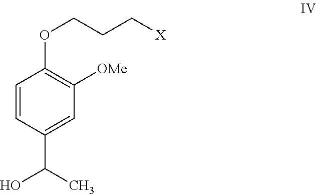

wherein X is as defined above; and

b) oxidizing the hydroxy compound of formula IV to produce the compound of formula II.

In accordance with another aspect of the present invention, there is provided an improved process for the preparation of iloperidone of formula I, comprising reacting the compound of formula II with benzisoxazole compound of formula V:

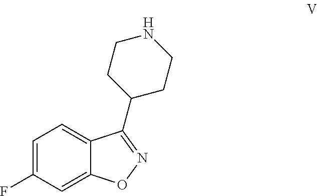

in the presence of an organic base to give iloperidone of formula I.

In accordance with another aspect of the present invention, there is provided a novel compound of formula IV:

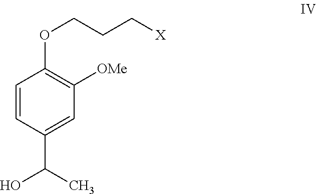

wherein X is selected from F, Br, Cl and I.

### DETAILED DESCRIPTION OF THE INVENTION

According to one aspect of the present invention, there is provided a novel process for preparation of intermediate of iloperidone of formula II:

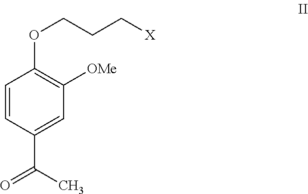

wherein X is selected from F, Br, Cl and I;
which comprises:

a) reacting an aldehyde compound of formula III:

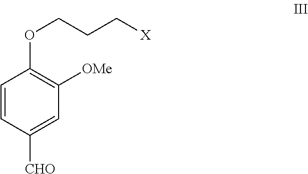

with methyl magnesium halide to produce a hydroxy compound of formula IV:

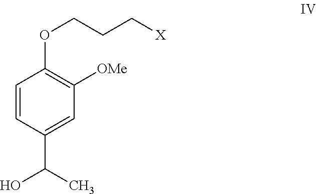

wherein X is as defined above; and

b) oxidizing the hydroxy compound of formula IV to produce the compound of formula II.

The quantity of methyl magnesium halide is not critical, but for better yield 1.5 to 2.5 molar equivalents of methyl magnesium halide per equivalent of the aldehyde compound of formula III is used.

In one embodiment, the reaction between the compounds of formula III and methyl magnesium halide is carried out in a solvent. Any solvent, which is neutral towards the reactants, may be used. Operable solvents include cyclic ethers such as tetrahydrofuran and methyl tetrahydrofuran; ethers such as diethyl ether; and aromatic solvents such as toluene and xylene; and a mixture thereof.

In another embodiment, the reaction is performed at or below boiling temperature of the solvent used; more preferably at 25° C. to the boiling temperature of the solvent used; and most preferably at the boiling temperature of the solvent used.

Time required for completion of the reaction depends on the factors such as the temperature at which the reaction is carried out.

After completion of the reaction, the reaction mass may be treated with a mixture of water and hydrochloric acid.

The product obtained may be used directly in the next step, or it can be isolated from the reaction mixture and then used in the next step, to produce the compound of formula II.

The oxidation reaction is performed using any oxidizing reagent commonly known for such purpose. Among them, chromic acid, potassium permanganate and aluminum isopropoxide are more preferred. More preferable oxidizing reagent is chromic acid.

In one embodiment, the oxidation reaction is preferably performed by contacting the hydroxy compound of formula IV with an oxidizing reagent in the presence of an aprotic solvent such as methylene dichloride, ethylene dichloride or chloroform; ethers such as diethyl ether or diisopropyl ether.

According to another aspect of the present invention, there is provided a novel process for the preparation of iloperidone of formula I:

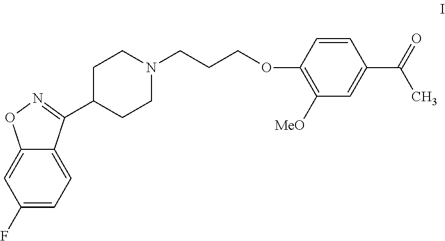

comprising reacting the compound of formula II:

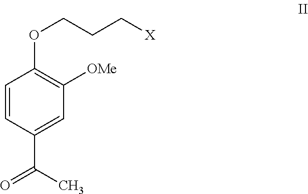

wherein X is selected from F, Br, Cl and I;
with benzisoxazole compound of formula V:

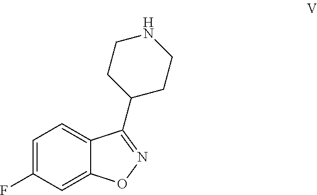

in the presence of an organic base to produce the iloperidone of formula I.

In one embodiment, the organic base is selected from the group consisting of N,N-diisopropylamine, tributylamine, N,N-dimethylaniline, 4-dimethylaminopyridine, ethyldiisopropylamine, N-ethylmorpholine, 2,4,6-trimethylpyridine and triethylamine; and a more preferable organic base is N,N-diisopropylamine or tributylamine. The reaction may be carried out in the presence of a solvent or the base used may also serve as a solvent. A most preferable solvent is methanol.

In another embodiment, the reaction is performed at a temperature of about 30° C. to the boiling temperature of the solvent used; and more preferably at 40° C. to 100° C.

Time required for completion of the reaction depends on the factors such as the solvent used and the temperature at which the reaction is carried out. For example, if the reaction is carried out by contacting the compound of formula II with benzisoxazole compound of formula V in N,N-diisopropylamine and methanol under reflux conditions, about 20 to 25 hours is required for the reaction completion.

According to another aspect of the present invention, there is provided a novel compound of formula IV:

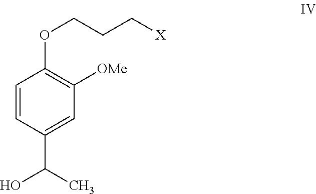

wherein X is selected from F, Br, Cl and I.

The following examples are given for the purpose of illustrating the present invention and should not be considered as limitations on the scope and spirit of the invention.

### Reference Example

To a stirred mixture of vanillin (50 gm, 0.33 moles), potassium carbonate (54.5 gm, 0.4 moles) and acetone (500 ml), 1-bromo-3-chloropropane (72.5 gm, 0.46 moles) was added for 30 minutes. The mixture was heated for 16 hours at reflux temperature. The reaction mass was filtered and the solvent was distilled off to produce a residue. The resulting residue was dissolved in methylene dichloride (100 ml), the organic layer was washed with water (50 ml) and then concentrated to produce a residue. The residue was triturated with n-hexane (50 ml) to produce 70 gm of 4-(3-chloropropoxy)-3-methoxybenzaldehyde (M.P—53° C. to 55° C.).

### EXAMPLES

### Example 1

To a mixture of magnesium metal turnings (10 gm, 0.42 moles) and ether (80 ml), a mixture of methyl iodide (33.6 ml, 0.54 moles) and ether (80 ml) was slowly added at ambient temperature, and then heated for 1 hour at reflux temperature. The reaction mass was cooled in an ice bath, followed by the addition of a mixture of 4-(3-chloropropoxy)-3-methoxybenzaldehyde (40 gm, 0.17 moles) and ether (280 ml) at 0 to 5° C. The mixture was heated for 6 hours at reflux temperature. The reaction mass was cooled to ambient temperature and then poured into ice (200 gm), water (50 ml) and dilute hydrochloric acid (30 ml) mixture. The organic layer was separated and the resulting aqueous layer was extracted with diisopropyl ether (2×200 ml). The combined organic layers were washed with 2% sodium bicarbonate solution (20 ml) and water (80 ml), followed by evaporation of the solvent to produce 40 gm of 1-[4-(3-chloropropoxy)-3-methoxyphenyl]ethanol.

### Example 2

To a stirred mixture of potassium dichromate (11.8 gm, 0.04 moles) and water (37 ml), sulfuric acid (9 ml, 0.16 mol) was slowly added at 20° C. 1-[4-(3-Chloropropoxy)-3-methoxyphenyl)ethanol (30 gm, 0.12 moles) was dissolved in ether (120 ml) and the resulting solution was slowly added to the above mixture at 10 to 15° C. The mixture was stirred for 2 hours at ambient temperature. The organic layer was separated from the reaction mass and the aqueous layer was extracted with ether (45 ml). The resulting extracts were combined and washed with 2% sodium bicarbonate solution (15 ml) and water (30 ml), followed by evaporation of the solvent to produce a residue. The resulting residue was triturated in diisopropyl ether (10 ml) to produce 16 gm of 1-[4-(3-chloropropoxy)-3-methoxyphenyl]ethanone. The resulting solid was recrystallized from ethyl alcohol (50 ml) to yield 11 gm of 1-[4-(3- chloropropoxy)-3-methoxyphenyl]ethanone (M. P: 57.5 to 58.5° C.).

### Example 3

Example 2 was repeated using methylene dichloride instead of ether to yield 10.8 gm of 1-[4-(3--chloropropoxy)-3-methoxyphenyl]ethanone (M. P: 57.6 to 58.4° C.).

### Example 4

1-[4-(3-Chloropropoxy)-3-methoxyphenyl]ethanone (20 gm, 0.08 moles) was dissolved in methanol (200 ml), the resulting solution was added to a mixture of 6-fluoro-3-(4-piperidinyl)-1,2-benzisoxazol (18 gm, 0.08 moles) and diisopropyl amine (25 gm, 0.25 moles). The resulting mixture was heated under reflux temperature for overnight and then concentrated under reduced pressure. The resulting crude product was dissolved in chloroform (200 ml), washed with water (40 ml) and then concentrated to produce crude iloperidone. Recrystallisation of the crude iloperidone from ethanol to produce 15.1 gm of pure iloperidone (M.P: 118 to 120° C.).

1. A process for the preparation of an iloperidone intermediate of formula II: 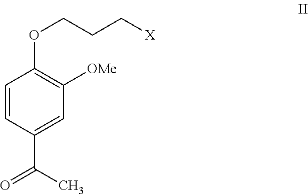
wherein X is selected from F, Br, Cl and I; comprising:
a) reacting an aldehyde compound of formula III: 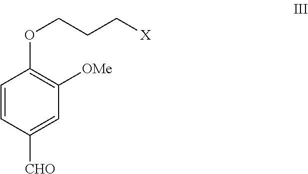
wherein X is as defined above;
with methyl magnesium halide to produce a hydroxy compound of formula IV: 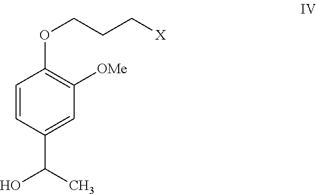
wherein X is as defined above; and
b) oxidizing the hydroxy compound of formula IV to produce the compound of formula II.

2. The process of claim 1, wherein the reaction in step-(a) is carried out in a solvent, wherein the solvent is selected from the group consisting of cyclic ethers, ethers, aromatic solvents, and mixtures thereof.

3. The process of claim 2, wherein the solvent is selected from the group consisting of tetrahydrofuran, methyl tetrahydrofuran, diethyl ether, toluene, xylene, and mixtures thereof.

4. The process of claim 1, wherein the reaction in step-(a) is carried out at below the boiling temperature of the solvent used; and wherein the oxidation reaction in step-(b) is performed by contacting the hydroxy compound of formula IV with an oxidizing reagent.

5. The process of claim 4, wherein the reaction in step-(a) is carried out at 25° C. to the boiling temperature of the solvent used; and wherein the oxidizing reagent used in step-(b) is selected from the group consisting of chromic acid, potassium permanganate and aluminum isopropoxide.

6. (canceled)

7. (canceled)

8. (canceled)

9. The process of claim 1, wherein the oxidation reaction is performed in the presence of an aprotic solvent.

10. The process of claim 9, wherein the aprotic solvent is selected from the group consisting of methylene dichloride, ethylene dichloride, chloroform, diethyl ether and diisopropyl ether.

11. A process for the preparation of iloperidone of formula I; 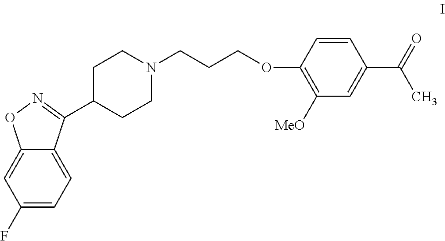
comprising reacting the compound of formula II: 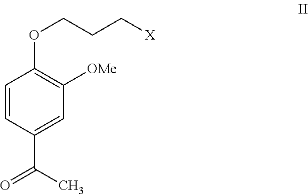
wherein X is selected from F, Br, Cl and I;
with a benzisoxazole compound of formula V: 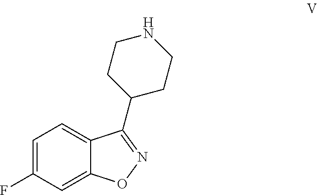
in the presence of an organic base to produce the iloperidone of formula I.

12. The process of claim 11, wherein the organic base is selected from the group consisting of N,N-diisopropylamine, tributylamine, N,N-dimethylaniline, 4-dimethylaminopyridine, ethyldiisopropylamine, N-ethylmorpholine, 2,4,6-trimethylpyridine and triethylamine; and wherein the reaction is carried out in presence of a solvent.

13. The process of claim 12, wherein the organic base is N,N-diisopropylamine or tributylamine; and wherein the solvent is methanol.

14. (canceled)

15. (canceled)

16. The process of claim 11, wherein the reaction is carried out at about 30° C. to the boiling temperature of the solvent used.

17. The process of claim 16, wherein the reaction is carried out at 40° C. to 100° C.

18. A novel compound of formula IV. 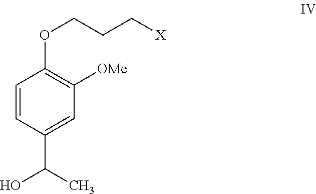
wherein X is selected from F, Br, Cl and I.
